(19) United States
(12) Patent Application Publication
(10) Pub. No.: US 2022/0195373 A1
(43) Pub. Date: Jun. 23, 2022

(54) INCUBATOR WITH A MAGNETICALLY-QUIET INCUBATOR CHAMBER AND METHODS OF MAKING AND USING

(71) Applicant: NearField Atomics Inc., Seattle, WA (US)

(72) Inventors: John T. Butters, Seattle, WA (US); Lisa C. Butters, Seattle, WA (US); George Smilov, Bellingham, WA (US); Jered L. Singleton, Bainbridge Island, WA (US); Philip J. Brock, North Bend, WA (US); John Thomas Sarlina, Seattle, WA (US); Ben Johannes Decock, Snohomish, WA (US)

(21) Appl. No.: 17/549,485

(22) Filed: Dec. 13, 2021

Related U.S. Application Data
(60) Provisional application No. 63/126,840, filed on Dec. 17, 2020.

(51) Int. Cl. C12M 1/00; C12M 1/34

(52) U.S. Cl. CPC C12M 41/14; C12M 23/34; C12M 41/40; C12M 29/26; C12M 41/34; C12M 23/38

(57) ABSTRACT

An incubator system includes an incubator unit including incubator chamber defined by chamber walls formed of a non-magnetic material; a control unit physically separated from the incubator unit and including operational controls for operation of the incubator system; and at least one duct coupling the incubator unit to the control unit, wherein the incubator system is configured so that the incubator chamber experiences a magnetic field variation of no more than 100 nT arising from the incubator system during incubation operation of the incubation system.

### CROSS-REFERENCE TO RELATED APPLICATIONS

This application claims the benefit under 35 U.S.C. § 119(e) of U.S. Provisional Patent Application Ser. No. 63/126,840, filed Dec. 17, 2020, which is incorporated herein by reference.

### FIELD

The present invention is directed to the area of incubators and incubator systems and methods of making and using. The present invention is also directed to incubators and incubator systems with a magnetically-quiet incubator chamber and methods of making and using.

### BACKGROUND

In microbiology, an incubator is an insulated and enclosed device that maintains conditions of temperature, humidity, and other environmental conditions required for the growth of organisms. Incubator environmental homogeneity facilitates reproducible cell culture results. However, Static and pulsed magnetic fields inside commercially available incubators create spatially differentiated magnetic environments for cell cultures along one or more of the x, y, and z axes.

BRIEF SUMMARY

One embodiment is an incubator system that includes an incubator unit including incubator chamber defined by chamber walls formed of a non-magnetic material; a control unit physically separated from the incubator unit and including operational controls for operation of the incubator system; and at least one duct coupling the incubator unit to the control unit, wherein the incubator system is configured so that the incubator chamber experiences a magnetic field variation of no more than 100 nT arising from the incubator system during incubation operation of the incubation system.

In at least some embodiments, the incubator unit further includes a housing disposed around the incubator chamber, wherein the incubator housing defines an open space between the chamber walls of the incubator chamber and the incubator housing, wherein the at least one duct is coupled to the incubator unit to supply or receive gas to or from the open space. In at least some embodiments, the control unit further includes a heater and fans for generating heated air to pass into the at least one duct for heating the incubator chamber or maintaining a temperature of the incubator chamber.

In at least some embodiments, the chamber wall of the incubator chamber provide passive magnetic field shielding for the incubator chamber. In at least some embodiments, the chamber walls of the incubator chamber are made of copper. In at least some embodiments, incubator unit includes a door to the incubator chamber and the chamber walls of the incubator chamber includes a door panel that is separate from the door of the incubator unit.

In at least some embodiments, the control unit includes electronic components and magnetic field shielding around at least a portion of the electronic components. In at least some embodiments, the magnetic field shielding includes passive magnetic field shielding made of a non-magnetic material that is disposed between the portion of the electronic components and the incubator unit. In at least some embodiments, the magnetic field shielding includes active magnetic field shielding for generating a magnetic field that counteracts a magnetic field generated by operation of the portion of the electronic components.

In at least some embodiments, the incubator system further includes magnetic field sensors disposed in the incubator chamber and coupled, or coupleable, to the control unit to monitor variation of a magnetic field within the incubator chamber. In at least some embodiments, the incubator system further includes at least one sensor for monitoring at least one of the following in the incubator chamber: temperature, humidity, gas composition, or gas pressure. In at least some embodiments, the incubator system is configured so that the incubator chamber experiences a magnetic field variation of no more than 10 nT arising from the incubator system during incubation operation of the incubation system.

Another embodiments is an incubator system that includes an incubator unit including incubator chamber defined by chamber walls formed of a non-magnetic material that provides passive magnetic field shielding for the incubator chamber; a control unit physically separated from the incubator unit and including operational controls for operation of the incubator system, electronic components for the operational controls, and magnetic field shielding around at least a portion of the electronic components; and at least one duct coupling the incubator unit to the control unit.

In at least some embodiments, the incubator system is configured so that the incubator chamber experiences a magnetic field variation of no more than 100 nT arising from the incubator system during incubation operation of the incubation system. In at least some embodiments, the magnetic field shielding of the control unit includes passive magnetic field shielding made of a non-magnetic material that is disposed between the portion of the electronic components and the incubator unit. In at least some embodiments, the magnetic field shielding of the control unit includes active magnetic field shielding for generating a magnetic field that counteracts a magnetic field generated by operation of the portion of the electronic components.

In at least some embodiments, the incubator system further includes magnetic field sensors disposed in the incubator chamber and coupled, or coupleable, to the control unit to monitor variation of a magnetic field within the incubator chamber. In at least some embodiments, the incubator system further includes at least one sensor for monitoring at least one of the following in the incubator chamber: temperature, humidity, gas composition, or gas pressure.

In at least some embodiments, the incubator unit further includes a housing disposed around the incubator chamber, wherein the incubator housing defines an open space between the chamber walls of the incubator chamber and the incubator housing, wherein the at least one duct is coupled to the incubator unit to supply or receive gas to or from the open space. In at least some embodiments, the control unit further includes a heater and fans for generating heated air to pass into the at least one duct for heating the incubator chamber or maintaining a temperature of the incubator chamber.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a block diagram of one embodiment of an incubator system, according to the invention;

FIG. 2 is another block diagram of an embodiment of an incubator system, according to the invention;

FIG. 3A is a side perspective view of one embodiment of an incubator system, according to the invention;

FIG. 3B is a front view of the incubator system of FIG. 3A, according to the invention;

FIG. 3C is a back view of the incubator system of FIG. 3A, according to the invention;

FIG. 3D is a back perspective view of the incubator system of FIG. 3A illustrating gas flow for heating or cooling, according to the invention;

FIG. 3E is a back perspective view of the incubator system of FIG. 3A illustrating a decontamination coil, according to the invention;

FIG. 3F is a front view of an incubator unit of the incubator system of FIG. 3A with an open door, according to the invention;

FIG. 3G is a front view of the incubator unit of the incubator system of FIG. 3A with individual compartment doors of an incubator chamber open, according to the invention;

FIG. 3H is a front view of the incubator unit of the incubator system of FIG. 3A with a door panel of the incubator chamber open, according to the invention; and

FIG. 3I is a close-up front view of the incubator chamber of the incubator system of FIG. 3A, according to the invention.

### DETAILED DESCRIPTION

The present invention is directed to the area of incubators and incubator systems and methods of making and using. The present invention is also directed to incubators and incubator systems with a magnetically-quiet incubator chamber and methods of making and using.

An incubator or incubator system with a magnetically-quiet incubator chamber is described herein. In the magnetically-quiet incubator chamber, magnetic field variations are reduced substantially when compared to commercially available incubators of a similar size or type. In at least some embodiments, the magnetic field variation, arising from operation of the incubator system, within the magnetically-quiet incubator chamber is no more than 100, 50, or 10 nT during incubation operation. In at least some embodiments, the magnetic field variation within the magnetically-quiet incubator chamber is no more than 100, 50, or 10 nT during incubation operation.

The incubator with a magnetically-quiet incubator chamber, as well as the design principles and features disclosed herein, are applicable to all incubator types and applications including, but not limited to, tissue culture incubators and applications, bioreactors within incubators, IVF (in vitro fertilization) incubators and applications, neonatal incubators and applications, and the like.

The separation of components and systems of the incubator that produce electromagnetic fields (EMF) from the incubation chamber facilitates reducing or eliminating spatially-differentiated EMF environments in the incubation chamber. This applies to both static and pulsed EMF. In at least some embodiments, the incubator enables frequency and electromagnetic experiments in an incubator chamber free of a differentiated static and pulsed EMF environment or with substantially reduced EMF compared to conventional incubators.

In at least some embodiments, the incubation chamber, the remote components and systems (e.g., a control unit as described below), the operator control/interface (e.g., a display as described below), or any combination thereof are shielded to prevent or reduce system and component EMF from entering the incubation chamber. The magnetic field (or EMF) shielding can be passive shielding or active shielding or any combination thereof. In at least some embodiments, the passive shielding can be multilayered shielding and can be a combination of different metals or metallic plastics. In at least some embodiments, the incubation chamber is disposed within shielding which may be multilayer shielding. Additionally or alternatively, the remote components, systems, operator control/interface, and other controls can be disposed within their own shielding (as a group, as subgroups, or individually, or any combination thereof) which may be multilayer shielding.

The incubation chamber can include one or more bioreactors, one or more cell cultures (including 2D or 3D cell cultures), one or more other reactors or systems (for example, cell, tissue bacteria reactors or systems), one or more chemical or drug testing environments, or the like or any combination thereof.

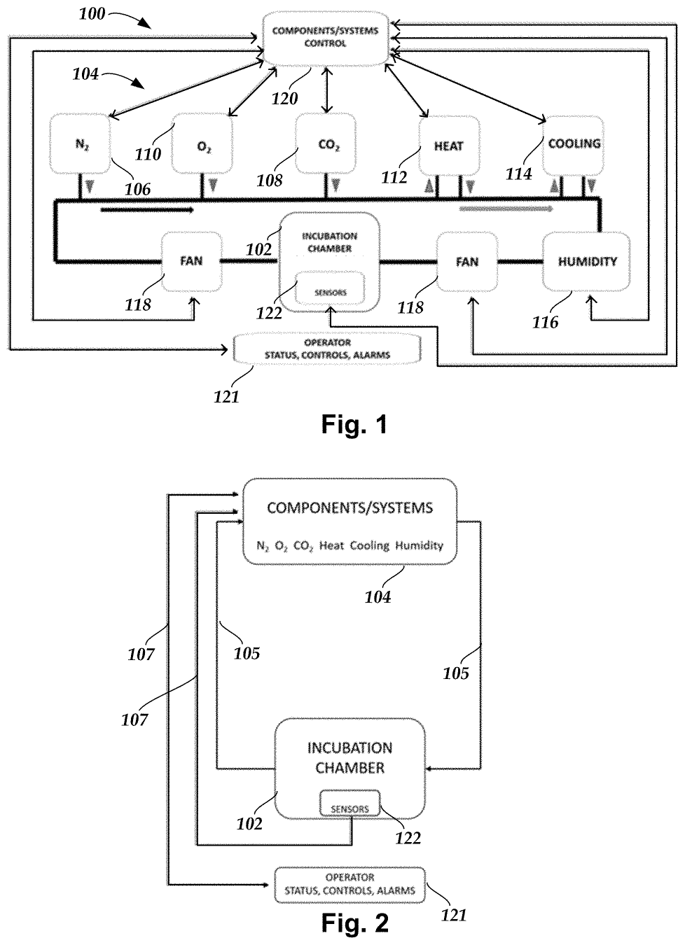

One or more systems and components of the incubator are positioned remote from the incubation chamber. FIG. 1 is a block diagram of one embodiment of an incubator system 100 with a magnetically-quiet incubator chamber 102. As illustrated in FIG. 1, various environmental controls and systems 104 are maintained outside of the incubator chamber. In at least some embodiments, the controls/systems 104 are provided in one or more control units that are physically separated from an incubator unit that includes the incubator chamber 102. These controls/systems 104 can include controls/systems 106 for N₂ (nitrogen) flow, controls/systems 108 for CO₂ (carbon dioxide) flow, controls/systems 110 for O₂ (oxygen) flow, controls/systems 112 for heating, controls/systems 114 for cooling, controls/systems 116 for controlling or monitoring humidity, and controls/systems for air flow (e.g., fans 118). In at least some embodiments, the incubator chamber 102 contains one or more sensors (for example, optical or magnetoresistive (MR) sensors) 122 to monitor incubator environment conditions, such as magnetic field variation, humidity, temperature, gas pressure or composition, or any combination thereof.

In at least some embodiments, these controls/systems can be manually controlled or controlled by one or more processors (optionally, with an artificial intelligence (AI) module) using a central control hub 120 (e.g., a control unit as described below). The central control hub may include a display 121 for presenting information (for example, settings, status, or warnings) to an operator, receiving instructions (for example, selecting or altering settings) from the operator, or for any other suitable use.

It will be understood that other incubator systems may include more or fewer controls/systems including, for example, more or fewer types of gas controls/systems. In at least some embodiments, the incubator enables magnetically-quiet incubation using gas combinations for hypoxia or hyperoxia environments or gas combinations for IVF (embryo growth) or for any other application or type of incubator system.

FIG. 2 illustrates the incubator chamber 102, the central control hub 120, and the display 121, as well as ducts 105 and control lines 107 between the incubator chamber and the central control hub. The ducts 105 can be made of any suitable materials (preferably, non-magnetic materials) and may also utilize any suitable insulation materials. The central control hub 120 and the display 121 are separated or remote from the incubator chamber 102 and, at least in some embodiments, the incubator chamber 102 is separated or remote from the other components and systems, as illustrated in FIGS. 1 and 2.

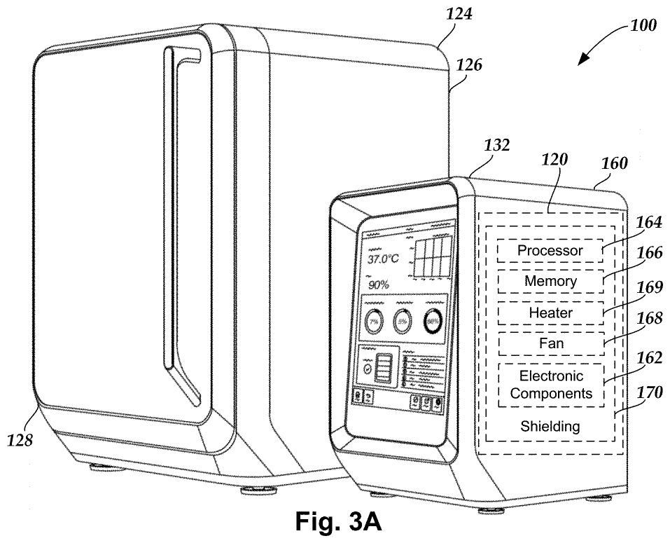

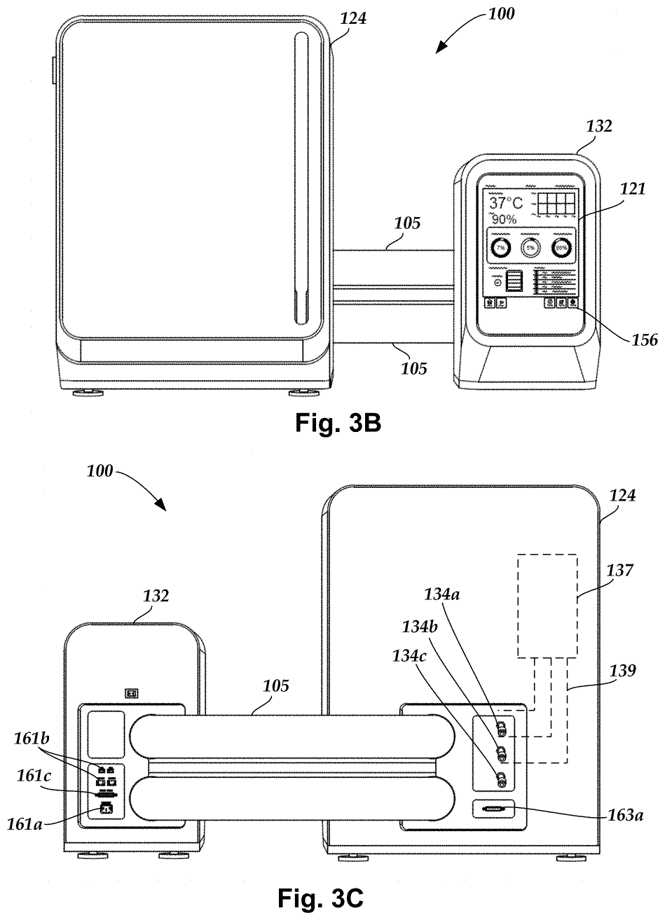

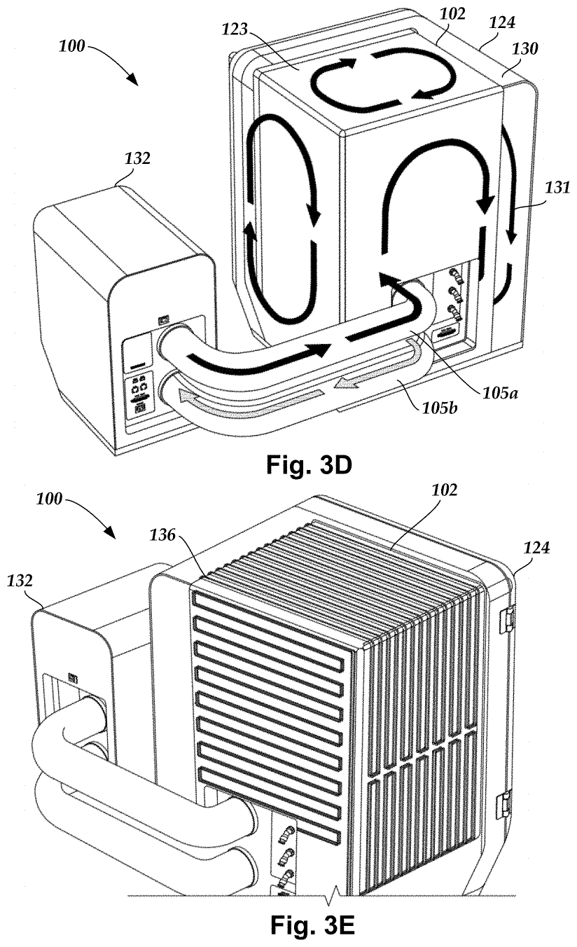

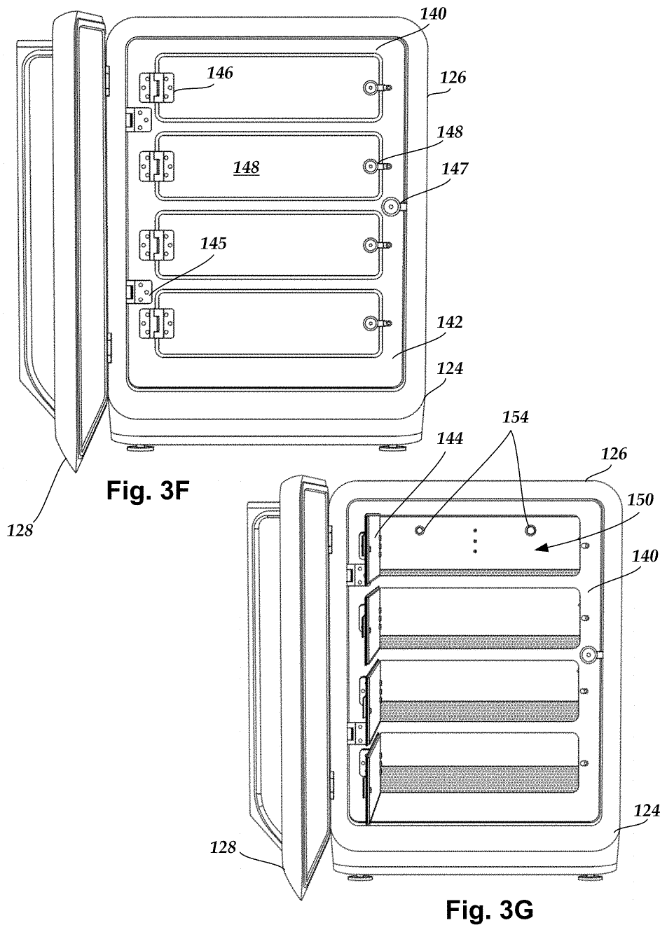

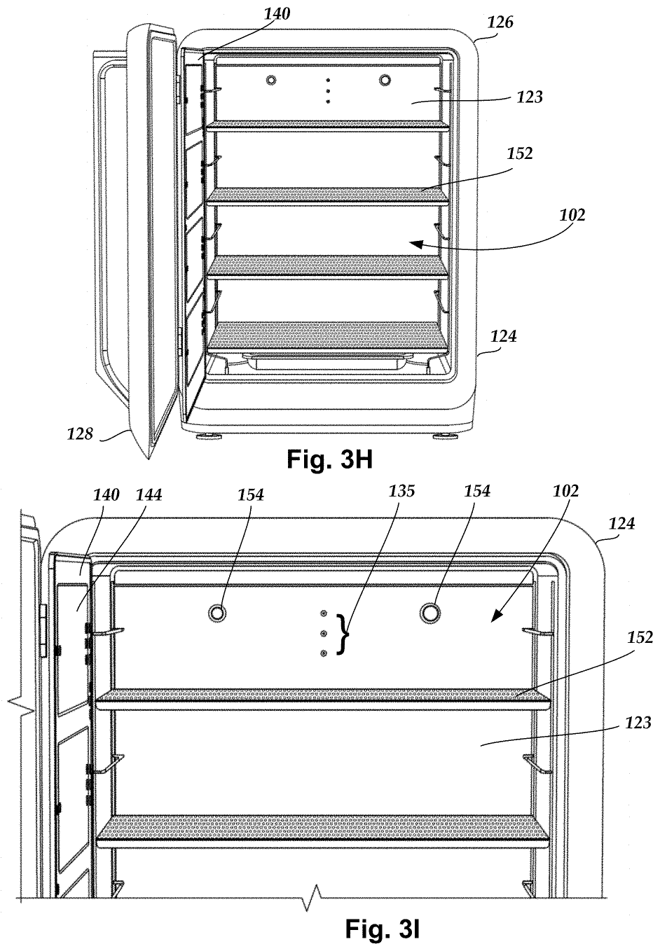

FIGS. 3A to 3I illustrate one embodiment of an incubator system 100 with an incubator unit 124 with an incubator chamber 102 (FIG. 3H) defined by chamber walls 123 (FIG. 3H), an incubator housing 126, and a door 128 (which may form, or include one or more panels forming, one of the chamber walls). The incubator system 100 also includes a control unit 132 that includes the central control hub 120 (FIG. 1) and a display 121. The incubator unit 124 and the control unit 132 are physically separated from each other to reduce magnetic fields generated by the control unit from being experienced in the incubator chamber 102. Ducts 105 couple the incubator unit 124 to the control unit 132.

In at least some embodiments, the incubator chamber 102 has non-magnetic chamber walls 123 (for example, chamber walls made of copper, copper alloy, aluminum, non-magnetic stainless steel, non-magnetic alloy, or other non-magnetic material) which may be seamless. In at least some embodiments, the incubator chamber 102 (or at least the chamber walls 123 of the incubator chamber) is not made of plastic materials as those materials may not be suitable to withstand heat decontamination. In at least some embodiments, the incubator chamber 102 may include a Faraday cage, mu-metal, or other passive magnetic field (or EMF) shielding. For example, in at least some embodiments, to provide magnetic field (or EMF) shielding the chamber walls 123 of the incubator chamber 102 are made of copper or other non-magnetic material which, preferably, forms a Faraday cage.

In at least some embodiments, as illustrated in FIG. 3F, one of the chamber walls 123 of the incubator chamber 102 forms a door panel 140 which may be separate from the door 128 of the incubator unit 124 or may be coupled (permanently or non-permanently) to the door of the incubator unit 124. In at least some embodiments, the door panel 140 may include a frame 142 (with associated hinges 145 and a fastener 147 to fasten the frame to the incubator housing 126 or the door 128 or any combination thereof) and one or more individual compartment doors 144 with associated hinges 146 and fasteners 148 (for fastening the compartment door to the frame). In other embodiments, the door panel 140 can be a single panel optionally with hinges 145 and a fastener 147 to fasten the door panel to the incubator housing 126 or the door 128 or any combination thereof.

In at least some embodiments, as illustrated in FIGS. 3G, 3H, and 3I, the incubator chamber 102 can be divided into multiple compartments 150 which can be divided by compartment dividers 152 (FIGS. 3H and 3I) that can be solid or mesh or any other suitable configuration. In at least some embodiments, the compartment dividers 152 are made of non-magnetic material (for example, copper or mu-metal). In at least some embodiments, the dividers 152 are removable or reconfigurable within the incubator chamber 102.

Returning to FIG. 3A, the control unit 132 includes a control unit housing 160, the central control hub 120, the display 121, and one or more input/output ports 161a, 161b, 161c (FIG. 3C). The central control hub 120 includes electronic components 162, at least one processor 164, at least one memory 166, and, optionally, at least one fan 168 for air flow (and may also act as cooling mechanism or there may be a separate cooling device), and a heater 169. (Dotted lines in the Figures indicate elements that are within the illustrated component.) As illustrated in FIG. 3C, the central control hub 120 is coupled to the input/output ports which can include, for example, one or more power ports 161a, one or more data/control ports 161b (e.g., USB or Ethernet ports), and at least one power/control port 161c that can be coupled via a cable (not shown) to a power/control port 163a on the incubator unit 124.

Returning to FIG. 3A, in at least some embodiments, the control unit 132 includes magnetic field (or EMF) shielding 170 which can be passive shielding (for example, shields made of non-magnetic material, such as copper or mu-metal) or active shielding (for example, one or more coils through which current can be passed to reduce or counteract the magnetic fields generated by the central control hub 120 or other components of the control unit 132) or any combination thereof. Any suitable, known active or passive shielding arrangement can be used as the shielding 170. The magnetic field (or EMF) shielding 170 can reduce the magnetic field generated by the components of the control unit 132 and, thereby, reduce the magnetic field variation experienced in the incubation chamber 102 of the incubation unit 124 which is separated from the control unit 132.

In at least some embodiments, as illustrated in FIG. 3D, the incubator unit 124 includes an open space 130 between the housing and one or more of the chamber walls 123 (for example, all of the chamber walls or all of the chamber walls except that chamber wall that forms the door panel 140 or the floor of the incubator chamber) that define the incubator chamber 102. Heated or cooled gas 131 (for example, air or nitrogen) can flow through the ducts 105 into the open space 130 to heat or cool the incubator chamber 102 or to maintain the temperature of the incubator chamber or any combination thereof. One of the ducts 105 can be a supply duct 105a and another one of the ducts can be a return duct 105b. In at least some embodiments, the control unit 132 includes a heater 169 and fans 168 for flow of hot air to heat the incubator chamber 102 or maintain a temperature of the incubator chamber. In at least some embodiments, the fans 168 (or an optional cooling device, not shown) can be used to flow cooler air to cool the incubator chamber 102 or maintain a temperature of the incubator chamber.

In at least some embodiments, as illustrated in FIG. 3E, the incubator unit 124 can include a heating coil 136 or other heating arrangement for decontamination of the incubator chamber 102. Typically, such decontamination does not occur during incubation operation and, at least in some embodiments, the decontamination operation may exceed the magnetic field variation thresholds. In at least some embodiments, the heating coil 136 is coupled to the control unit 132 via the power/control ports 161c, 163a and the control unit can initiate, control, or halt the decontamination operation.

In at least some embodiments, the incubator unit 124 includes gas nozzles 134 for attachment of gas sources for gases such as, for example, oxygen, carbon dioxide, nitrogen, or any other suitable gas. The gas nozzles 134 are coupled to conduits 139 that extend to outlets 135 (FIG. 3I) into the incubator chamber 102.

In at least some embodiments, the gas sources (not shown) include regulators, valves, or other mechanisms for controlling the flow of the particular gas. In at least some embodiments, one or more sensors 154 (FIG. 3G) can be used to monitor gas flow or gas composition in the incubator chamber 102. In at least some embodiments, the incubation system 100 may warn or provide a message to a user if the gas flow or gas composition is outside preset thresholds. In at least some embodiments, the control unit 132 can be coupled to regulators, valves, or other mechanisms associated with the gas source(s) to monitor or control the gas flow.

In at least some embodiments, the control unit 132 can control the mixture of gases, as well as the pressure, of the atmosphere within the incubator chamber 102. In at least some embodiments, regulators or valves 137 can be disposed in the incubator unit 124 and controllable by the control unit 132 through signals provided via the power/control ports 161c, 163a to control the flow of the gas into the incubator chamber 102. In at least some embodiments, the regulators or valves 137 include passive (or active) shielding to reduce or prevent magnetic fields generated by the regulators or valves.

In at least some embodiments, as illustrated in FIG. 3G, one or more sensors 154 are provided in the incubator chamber 102. In at least some embodiments, the one or more sensors send (and optionally receive) signals to (and optionally from) the control unit 132 via the power/control ports 161c, 163a. Examples of suitable sensors 154 include, but are not limited to, temperature sensors, gas sensors, pressure sensors, magnetic field sensors, humidity sensors, or the like or any combination thereof. The sensors 154 can be any suitable type of sensor including, but not limited to, optical sensors, thermistors, magnetoresistive sensors, thermal conductive sensors, infrared sensors, nondispersive infrared (NDIR) sensors, zirconia, or the like or any combination thereof.

As an example, in at least some embodiments, two or more magnetoresistive sensors can be disposed at different sites in the incubator chamber 102 to monitor the magnetic field variation within the incubator chamber 102. In at least some embodiments, the magnetic field variation, arising from operation of the incubator system 100, within the magnetically-quiet incubator chamber 102 is no more than 100, 50, or 10 nT during incubation operation (preferably measured with the door 128 closed). In at least some embodiments, the magnetic field variation within the magnetically-quiet incubator chamber 102 is no more than 100, 50, or 10 nT during incubation operation (preferably measured with the door 128 closed). It will be recognized that the incubator system can include operations or states other than the incubation operation including, but not limited to, the off state (in which the incubator system 100 or control unit 132 is turned off) or the decontamination operation (during which the incubator chamber 102 is being decontaminated) or the like or any combination thereof. Another state is a calibration or post-decontamination state which can include activities such as, for example, one or more of the following: sensor calibration (e.g., calibration of the magnetoresistive sensors) or establishment of temperature, humidity, and gas (single gas or multiple gases) levels. In at least some embodiments, one or more of the sensors (for example, the magnetoresistive sensors) are removed for decontamination to, for example, avoid damage to the sensors.

In at least some embodiments, one or more optical or other sensors can be used to monitor temperature. In at least some embodiments, one or more optical or other sensors can be used to monitor humidity. In at least some embodiments, one or more zirconia, ultrasonic, thermal conductivity, infrared, NDIR, or other sensors can be used to monitor the gas composition or gas pressure.

The control unit 132 includes a display 121. The display 121 can be used to display information for an operator such as one or more of the following: a temperature of the incubator chamber 102, a relative humidity of the incubator chamber, an amount or percentage of each gas in the incubator chamber, a measure of the magnetic field variation in the incubator chamber, an indication whether the magnetic field variation is less than (or no more than) a threshold value, or a portion of a sensor log with information from the sensor(s) 154, or the like or any combination thereof.

In at least some embodiments, the processor 164 of the control unit 132 can identify circumstances in which a warning or alarm is to be displayed. Non-limiting examples of warnings or alarms can include a magnetic field variation that exceeds a threshold value, temperature or relative humidity changes that exceed a threshold variation value, gas composition changes that exceed a threshold variation value, loss of power, loss of connection to the incubation chamber 102, or the like or any combination thereof. In at least some embodiments, the warning or alarm can be visually displayed on the display 121, can include an auditory sound, or any combination thereof. In at least some embodiments, the display 121 can be a touchscreen display with controls 156 (FIG. 3B) that permit the operator to makes changes to the operation of the incubation system 100 such as one or more of the following: to turn the incubation system on or off, to set or change the temperature of the incubation chamber 102, to set or change the amount or percentage of each gas for the incubation chamber, to set or change a threshold for the magnetic field variation, to view or review the sensor log, to change settings, to set warnings, to save data to an external memory, to erase or write data internally, or the like or any combination thereof. In addition to, or an alternative to, the touchscreen display, other input arrangements or devices can be used for operator input including, but not limited to, buttons, switches, wireless or wired input devices (for example, a keyboard, mouse, or trackpad), or the like or any combination thereof.

In at least some embodiments, the incubation system 100 can collect and store operational and sensor data in the memory 166 or elsewhere. Examples of such data include, but are not limited to, the door opening or closing; measurement from the sensors 154; values for the temperature, humidity, or gas composition; or the like or any combination thereof. In at least some embodiments, the incubation system 100 may incorporate remote monitoring, cloud storage, or integration with other equipment or devices, or any combination thereof.

The above specification provides a description of the invention and the manufacture and use of the invention. Since many embodiments of the invention can be made without departing from the spirit and scope of the invention, the invention also resides in the claims hereinafter appended.

What is claimed as new and desired to be protected is:

1. An incubator system, comprising:
an incubator unit comprising incubator chamber defined by chamber walls formed of a non-magnetic material;
a control unit physically separated from the incubator unit and comprising operational controls for operation of the incubator system; and
at least one duct coupling the incubator unit to the control unit,
wherein the incubator system is configured so that the incubator chamber experiences a magnetic field variation of no more than 100 nT arising from the incubator system during incubation operation of the incubation system.

2. The incubator system of claim 1, wherein the incubator unit further comprises a housing disposed around the incubator chamber, wherein the incubator housing defines an open space between the chamber walls of the incubator chamber and the incubator housing, wherein the at least one duct is coupled to the incubator unit to supply or receive gas to or from the open space.

3. The incubator system of claim 2, wherein the control unit further comprises a heater and fans for generating heated air to pass into the at least one duct for heating the incubator chamber or maintaining a temperature of the incubator chamber.

4. The incubator system of claim 1, wherein the chamber wall of the incubator chamber provide passive magnetic field shielding for the incubator chamber.

5. The incubator system of claim 1, wherein the chamber walls of the incubator chamber are made of copper.

6. The incubator system of claim 1, wherein incubator unit comprises a door to the incubator chamber and the chamber walls of the incubator chamber comprises a door panel that is separate from the door of the incubator unit.

7. The incubator system of claim 1, wherein the control unit comprises electronic components and magnetic field shielding around at least a portion of the electronic components.

8. The incubator system of claim 7, wherein the magnetic field shielding comprises passive magnetic field shielding made of a non-magnetic material that is disposed between the portion of the electronic components and the incubator unit.

9. The incubator system of claim 7, wherein the magnetic field shielding comprises active magnetic field shielding for generating a magnetic field that counteracts a magnetic field generated by operation of the portion of the electronic components.

10. The incubator system of claim 1, further comprising a plurality of magnetic field sensors disposed in the incubator chamber and coupled, or coupleable, to the control unit to monitor variation of a magnetic field within the incubator chamber.

11. The incubator system of claim 1, further comprising at least one sensor for monitoring at least one of the following in the incubator chamber: temperature, humidity, gas composition, or gas pressure.

12. The incubator system of claim 1, wherein the incubator system is configured so that the incubator chamber experiences a magnetic field variation of no more than 10 nT arising from the incubator system during incubation operation of the incubation system.

13. An incubator system, comprising:
an incubator unit comprising incubator chamber defined by chamber walls formed of a non-magnetic material that provides passive magnetic field shielding for the incubator chamber;
a control unit physically separated from the incubator unit and comprising operational controls for operation of the incubator system, electronic components for the operational controls, and magnetic field shielding around at least a portion of the electronic components; and
at least one duct coupling the incubator unit to the control unit.

14. The incubator system of claim 13, wherein the incubator system is configured so that the incubator chamber experiences a magnetic field variation of no more than 100 nT arising from the incubator system during incubation operation of the incubation system.

15. The incubator system of claim 13, wherein the magnetic field shielding of the control unit comprises passive magnetic field shielding made of a non-magnetic material that is disposed between the portion of the electronic components and the incubator unit.

16. The incubator system of claim 13, wherein the magnetic field shielding of the control unit comprises active magnetic field shielding for generating a magnetic field that counteracts a magnetic field generated by operation of the portion of the electronic components.

17. The incubator system of claim 13, further comprising a plurality of magnetic field sensors disposed in the incubator chamber and coupled, or coupleable, to the control unit to monitor variation of a magnetic field within the incubator chamber.

18. The incubator system of claim 13, wherein the incubator unit further comprises a housing disposed around the incubator chamber, wherein the incubator housing defines an open space between the chamber walls of the incubator chamber and the incubator housing, wherein the at least one duct is coupled to the incubator unit to supply or receive gas to or from the open space.

19. The incubator system of claim 18, wherein the control unit further comprises a heater and fans for generating heated air to pass into the at least one duct for heating the incubator chamber or maintaining a temperature of the incubator chamber.

20. The incubator system of claim 13, further comprising at least one sensor for monitoring at least one of the following in the incubator chamber: temperature, humidity, gas composition, or gas pressure.
